(19) United States
(12) Patent Application Publication
(10) Pub. No.: US 2023/0166124 A1
(43) Pub. Date: Jun. 1, 2023

(54) INTRACAVITARY RADIOTHERAPY APPARATUS AND USE METHOD THEREOF

(71) Applicant: NANJING RONGSHENG MEDICAL TECHNOLOGY CO., LTD., Nanjing (CN)

(72) Inventors: Yonghua DONG, Solon, OH (US); Gaojun TENG, Nanjing (CN); Jiansong JI, Nanjing (CN); Huaqing YIN, Nanjing (CN); Haidong ZHU, Nanjing (CN); Jinhe GUO, Nanjing (CN)

(73) Assignee: NANJING RONGSHENG MEDICAL TECHNOLOGY CO., LTD., Nanjing, JS (CN)

(21) Appl. No.: 18/161,831

(22) Filed: Jan. 30, 2023

Related U.S. Application Data
(63) Continuation of application No. PCT/CN2021/108969, filed on Jul. 28, 2021.

(30) Foreign Application Priority Data
Jul. 28, 2020 (CN) 202010739356.2

(51) Int. Cl. A61N 5/10

(52) U.S. Cl. CPC A61N 5/1014; A61N 2005/1024; A61N 2005/1018

(57) ABSTRACT

The present invention discloses an intracavitary radiotherapy apparatus, which is used for carrying radioactive particles or particle strips, comprising: a body that is formed by winding metal wires, and is a wire mesh having a hollow cavity penetrating through same in the forward and backward directions; and a radioactive particle groove that is provided on the outer surface of the body, is a cylindrical wire mesh having a hollow cavity penetrating through same in the forward and backward directions, and is used for accommodating the radioactive particles or particle strips. The radioactive particle groove comprises a plurality of groove bodies arranged in parallel, and the spacing between two adjacent groove bodies is less than the length of the radioactive particles or particle strips.

### BACKGROUND

### Technical Field

The present invention relates to an intracavitary radiotherapy apparatus and a use method therefor, belonging to the field of radiotherapy instruments.

### Related Art

Intracavitary radiotherapy refers to a method for entering a focus part through a natural cavity (such as vagina, rectum, esophagus, trachea, and bronchus) of a human body and then introducing a radiation source into a tumor part for radiotherapy. Targeted local radiotherapy will be carried out while expanding by a stent, thereby not only reducing the toxic and side effects of the radiotherapy on the whole body, but also having better effect on the therapy.

An existing intracavitary radiotherapy apparatus is provided with a radioactive particle filling capsule on the surface of a reticular framework structure, and the radioactive particle filling capsule is capable of clamping and positioning radioactive particles through barbs (relative to the embedding direction) on the surface of the reticular framework structure or fixing and positioning the radioactive particles in a sewing mode.

However, in the existing intracavitary radiotherapy apparatus, the radioactive particle filling capsule is that the radioactive particles are arranged on the stent (the number and positions of the particles are fixed) in advance and then released into the human body. It may cause large size of the stent loaded with the particles, and complicate the implantation. Moreover, the radioactive particles are loaded in advance, so the positions of the radioactive particles cannot be correspondingly adjusted according to the individual state of illness of a patient, and it is difficult to accurately position the radioactive particles at the optimal position for intracavitary radiotherapy. Furthermore, an existing capsule filling or binding intracavitary radiotherapy apparatus cannot realize full-automatic production or has a complex production process, and therefore the production efficiency is low. Therefore, the existing intracavitary radiotherapy apparatus has high manufacturing cost and is not favorable for wide application.

### SUMMARY

The first technical problem to be solved by the present invention is to provide an intracavitary radiotherapy apparatus.

Another technical problem to be solved by the present invention is to provide a method for using the intracavitary radiotherapy apparatus.

In order to achieve above purposes, the present invention adopts the following technical solutions:

In a first aspect, an embodiment of the present invention provides an intracavitary radiotherapy apparatus, which is used for carrying radioactive particles or particle strips, including:

a body that is formed by winding metal wires and is a wire mesh having a hollow cavity penetrating through same in the forward and backward directions; and

a radioactive particle groove that is formed in the outer surface of the body,

is a cylindrical wire mesh having a hollow cavity penetrating through same in the forward and backward directions, and is used for accommodating the radioactive particles or particle strips, where

the radioactive particle groove includes a plurality of groove bodies arranged in parallel, and spacing between every two adjacent groove bodies is less than the length of the radioactive particles or the particle strips; and

the radioactive particle groove and the body are made of the same material by winding metal wires, or are made of biodegradable material by means of one-step injection molding.

Preferably, the radioactive particle groove is formed by inwards or outwards hot-pressing from the outer surface of the body after winding.

Preferably, the spacing between every two adjacent groove bodies is the same as that between body units.

Preferably, the diameter of the radioactive particle grooves is 0.8-1.2 times that of the radioactive particles.

Preferably, the radioactive particle groove is formed in the surface of the body in a protruding mode, and the inner diameter of the radioactive particle grooves is less than or equal to the diameter of the radioactive particles or particle strips.

Preferably, the radioactive particle groove is formed in the surface of the body in a concave mode, and the inner diameter of the radioactive particle grooves is less than or equal to the diameter of the radioactive particles or particle strips.

Preferably, the number of the radioactive particle grooves is different in the axial direction of the intracavitary radiotherapy apparatus.

Preferably, the intracavitary radiotherapy apparatus further includes guide wires, and the guide wires are fixed to the body or the radioactive particle grooves and are of a single-wire or double-wire structure.

In a second aspect, an embodiment of the present invention provides a method for using the intracavitary radiotherapy apparatus, including the following steps:

S1: placing the intracavitary radiotherapy apparatus at a target position in a human body;

S2: pushing a release catheter carrying radioactive particles or particle strips into the radioactive particle groove;

S3: pushing the radioactive particles or particle strips into the radioactive particle groove from the release catheter; and

S4: withdrawing the release catheter from the radioactive particle groove.

Preferably, the radioactive particle groove at least includes a first radioactive particle groove and a second radioactive particle groove.

In the steps S1-S4, the radioactive particles are placed in the first radioactive particle groove; then, the release catheter is pushed into the second radioactive particle groove; and the steps S3-S5 are repeated until all the radioactive particles or particle strips are placed in the corresponding radioactive particle grooves and the release catheter is withdrawn.

Preferably, the use method further includes the following step: guiding the release catheter to enter the radioactive particle groove by the guide wire arranged in the radioactive particle groove.

Preferably, the guide wire is of a single-wire structure connected with the radioactive particle groove or a double-wire structure penetrating through a shrinkage groove body.

Compared with the prior art, the intracavitary radiotherapy apparatus provided by the present invention is provided with the radioactive particle groove integrated with the body, has a more slender structure to cause a smaller wound, and can also enter a seriously blocked blood vessel; and compared with a stent pre-loaded with particles in the prior art, the intracavitary radiotherapy apparatus is more flexible, so that the compliance is improved. In addition, because the stent is placed in the human body firstly, then the radioactive particles can be accurately placed according to CT and other image display and the focus condition around the stent, both the number and the positions of the radioactive particles can be freely adjusted by a doctor according to an image, so that the number and the positions of the radioactive particles can be more accurate. Moreover, the manufacturing cost of the intracavitary radiotherapy apparatus can be reduced by the present invention. Due to the integrated design, sewing or welding or other process steps in the prior art are omitted, and therefore the manufacturing cost is reduced.

### BRIEF DESCRIPTION OF THE DRAWINGS

FIG. 1 is a three-dimensional structure schematic diagram of an intracavitary radiotherapy apparatus provided by an Embodiment I of the present invention;

FIG. 2 is a cross section schematic diagram perpendicular to an X axis of an intracavitary radiotherapy apparatus provided by an Embodiment I of the present invention;

FIG. 3 is a cross section schematic diagram perpendicular to an X axis of an intracavitary radiotherapy apparatus provided by an Embodiment II of the present invention;

FIG. 4 is a three-dimensional structure schematic diagram of an intracavitary radiotherapy apparatus provided by an Embodiment III of the present invention;

FIG. 5 is a cross section schematic diagram perpendicular to an X axis of an intracavitary radiotherapy apparatus provided by an Embodiment III of the present invention;

FIG. 6 is a three-dimensional structure schematic diagram of an intracavitary radiotherapy apparatus provided by an Embodiment IV of the present invention;

FIG. 7 is a three-dimensional structure schematic diagram of an intracavitary radiotherapy apparatus provided by an Embodiment V of the present invention;

FIG. 8 is a deformation example schematic diagram of an Embodiment V of the present invention; and

FIG. 9 is another deformation example schematic diagram of an Embodiment V of the present invention.

### DETAILED DESCRIPTION

The technical solutions of the present invention will be further described in detail with the accompanying drawings and specific embodiments.

An intracavitary radiotherapy apparatus provided by an embodiment of the present invention includes a body and a radioactive particle groove, where radioactive particles for radiotherapy can be arranged in the radioactive particle groove. The present invention is suitable for a biliary stent, a cervical stent, an esophageal stent, and the like.

Specifically, the body is formed by winding nickel-titanium alloy wires and is roughly a cylindrical wire mesh having a hollow cavity penetrating through same in the forward and backward directions. The body can also be made of titanium alloy and other verified metal materials available in a human body, or be made of a resin material such as PLA, and is simply directly formed in an injection molding mode, rather than in a winding or weaving mode.

The radioactive particle groove is formed in the surface of the body and is molded with the body by hot-pressing, and the radioactive particle groove is a cylindrical wire mesh having a hollow cavity penetrating through same in the forward and backward directions. Cross sections of the body and the radioactive particle groove are a part of a circle; and at least one or more radioactive particle grooves can be provided, and the number of the radioactive particle grooves can be selected according to actual needs or limiting conditions. The cross section of the radioactive particle groove can be in any shape, and can be randomly selected according to actual needs on the premise that the radioactive particles can be fixed.

The diameter of the section of the radioactive particle groove is greater than that of the radioactive particles. The width between every two wires forming the radioactive particle groove is less than the length of the radioactive particles. The metal wires adopted by the body and the radioactive particle groove are made of any one and/or more of nickel-titanium alloy, copper alloy and iron alloy.

The intracavitary radiotherapy apparatus provided by the embodiments of the present invention is provided with the radioactive particle groove integrally formed with the body, and the radioactive particle can be well fixed by the radioactive particle groove; the problem that the radioactive particles are prone to falling off when the intracavitary radiotherapy apparatus is placed in the human body is effectively solved; and radiotherapy can be accurately conducted in the preset body.

### Embodiment I

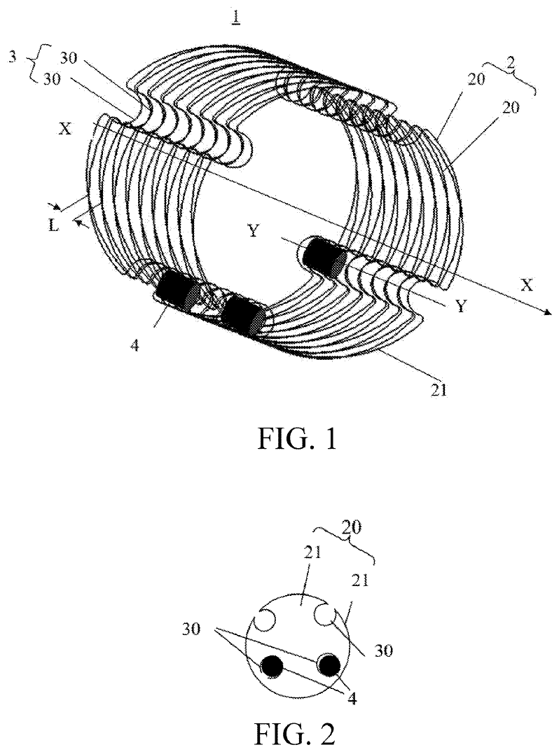

As shown in FIG. 1, a biliary stent is taken as an example in the Embodiment I of the present invention for introducing an intracavitary radiotherapy apparatus 1 provided by the present invention. The intracavitary radiotherapy apparatus 1 includes a body 2 and a radioactive particle groove 3, where radioactive particles 4 used for radiotherapy are arranged in the radioactive particle groove 3.

Specifically, the body 2 is formed by winding nickel-titanium alloy wires, is roughly a cylindrical wire mesh having a hollow cavity penetrating through same in the forward and backward directions, and has an axis X. Referring to FIG. 2, the body 2 includes a plurality of body parts 20 in the axial direction, and each body part 20 is formed by 4 arc-shaped nickel-titanium alloy wire body units 21 with the same radius in a roughly enclosing mode to form a circle. The plurality of body parts 20 are arranged in parallel (roughly in parallel) along the axis X of the body 2. In other words, in the cross section shown in FIG. 2, the body units 21 are ¼ arc sections, and the four body units 21 form an unclosed circular body part 20 in an enclosing mode. The plurality of body parts 20 are arranged in parallel along the axis X to form the body 2.

The description above is just for understanding easily. In fact, the whole intracavitary radiotherapy apparatus 1 is formed by weaving one or more wires, so the body units 21 are divided into two groups, one group including the body parts 20 which take an axis X as an axis and are inclined and arranged in parallel toward the front of the X axis (the right in the figure), and the other group including the body parts 20 which take the axis X as the axis and are inclined and arranged in parallel toward the rear of the X axis (the left in the figure). The two groups of body parts 20 are crossed to form a rhombus (the width is D) in FIG. 1.

The radioactive particle groove 3 and the body 2 can be formed by weaving one or more nickel-titanium alloy wires, and are formed by hot pressing from the outer surface of the body 2 to the inside (towards the axis direction). Specifically, a cylindrical wire mesh is woven by the metal wires; and then, a hot pressing process is carried out to apply pressure from the outer side of the cylindrical wire mesh to the inner side of the cylindrical wire mesh so as to form a plurality of radioactive particle grooves 3 by pressing. By adopting this manufacturing method, each groove body 30 of the radioactive particle grooves 3 is connected with the corresponding body unit 21 of the body 2 in a one-to-one correspondence mode. The corresponding connection in the embodiment refers to that each end of the groove body 30 is connected with a corresponding body unit 21. A situation that one end of the groove body 30 is connected with a plurality of body units 21, or one end of the body unit 21 is connected with a plurality of groove bodies 30 cannot occur. The difference from the prior art is that the radioactive particle groove 3 is wrapped by a flexible material, and the radioactive particles are attached to a stent wire in the prior art.

The radioactive particle groove 3 is connected with the body 2 and is roughly a cylindrical wire mesh having a hollow cavity penetrating through same in the forward and backward directions along the axis Y. The axis of each radioactive particle groove 3 is parallel to that of the body 2. Each radioactive particle groove 3 includes a plurality of groove bodies 30. The plurality of groove bodies 30 are arranged in parallel along the axis Y. As shown in FIG. 2, each groove body 30 can be a semicircle or a ¾ circle. Each groove body 30 is connected with two adjacent body units 21; and 4 groove bodies 30 are connected with 4 body units 21 at intervals to form a closed ring perpendicular to an axis X. The 4 radioactive particle grooves 3 can be uniformly distributed on the circumference formed by the body 2 (as shown in FIG. 1 and FIG. 2, the 4 groove bodies 30 are uniformly distributed on the circumference formed by the body parts 20), and can also be non-uniformly distributed (the 4 groove bodies 30 are non-uniformly distributed on the circumference formed by the body part 20).

In addition, the radioactive particle groove 3 is formed in the surface of the body 2 in a concave mode. The cross section of the vertical X axis of the intracavitary radiotherapy apparatus 1 provided by the embodiment is shown in FIG. 2. In the same cross section, the intracavitary radiotherapy apparatus includes 4 body units 21 and 4 groove bodies 30. Any one radioactive particle groove 3 can be internally provided with one or more radioactive particles 4.

The diameter of the radioactive particle groove 3 is equivalent to that of the radioactive particles 4 (equal to or slightly less than, or slightly greater than the diameter of the radioactive particles, for example, 0.01 mm); the diameter of the radioactive particle groove 3 just allows the radioactive particles to penetrate through, but cannot be too large, and most preferably, the diameter of the radioactive particle groove 3 is 0.8-1.2 times that of the radioactive particles 4. Therefore, the radioactive particles can be prevented from getting loose and deviating from an expected position.

As shown in FIG. 1, spacing L is formed between the body units 21 which are adjacently arranged along the axis X in the axial direction. As shown in FIG. 1, the body 2 and the radioactive particle groove 3 form a mesh composed of rhombuses. The width of the widest part of each rhombus part, namely the spacing L, is less than the length of the radioactive particles 4, so that a situation that the radioactive particles 4 disconnect from gaps of the rhombuses can be prevented. More preferably, the spacing L is less than half of the length of the radioactive particles 4. In other words, the length of the radioactive particles 4 is greater than 2 times of the spacing L, and in FIG. 1, the length of the radioactive particles 4 is greater than the width of the two rhombuses. Because the intracavitary radiotherapy apparatus is formed by weaving one metal wire, the spacing between every two adjacent groove bodies 30 is the same as that between the body units 21 and is L.

In addition, the radioactive particles 4 placed in the radioactive particle groove 3 are preferably placed at intersection parts of adjacent groove bodies 30, so that the radioactive particles 4 can be fixed more firmly.

As shown in FIG. 1, different numbers of radioactive particles 3 can be placed in 4 radioactive particle grooves 3 of the intracavitary radiotherapy apparatus 1 according to the design of the radiation dosimetry. For example, 2 radioactive particles are placed in one radioactive particle groove 3, and 1 radioactive particle is placed in the other radioactive particle groove 3. Therefore, the radiation dose can be conveniently controlled.

Moreover, the positions of the radioactive particles 3 in each radioactive particle groove 3 can be changed according to the design of the radiation dosimetry. For example, a plurality of radioactive particles 3 in the same radioactive particle groove 3 are continuously placed (adjacent radioactive particles are placed end to end), or a plurality of radioactive particles 3 in the same radioactive particle groove 3 are placed at intervals (a large gap is formed between adjacent radioactive particles, and the radioactive particles are discontinuous). Through such position design, the radioactive particles are placed at intervals for treating diffuse tumors; and the radioactive particles are continuously placed for treating exogenous tumors.

According to the intracavitary radiotherapy apparatus provided by the present invention, due to the design of radioactive particle groove in channel mode, both the number and positions of the radioactive particles placed in the radioactive particle groove can be conveniently adjusted by a doctor before an interventional operation, so that compared with the design that the positions and number of the radioactive particles are fixed when a stent leaves a factory in the prior art, the intracavitary radiotherapy apparatus is more suitable for various focus distribution conditions.

### Embodiment II

In the intracavitary radiotherapy apparatus 1 in Embodiment I, all radioactive particle grooves 3 are uniformly distributed in the Y-axis direction. In other words, groove bodies 30 distributed in parallel in the Y-axis direction are the same, and the sections, perpendicular to the X axis, of the intracavitary radiotherapy apparatus 1 are the same (FIG. 2).

However, in the embodiment, any radioactive particle groove 3 can be non-uniformly distributed in the axial direction.

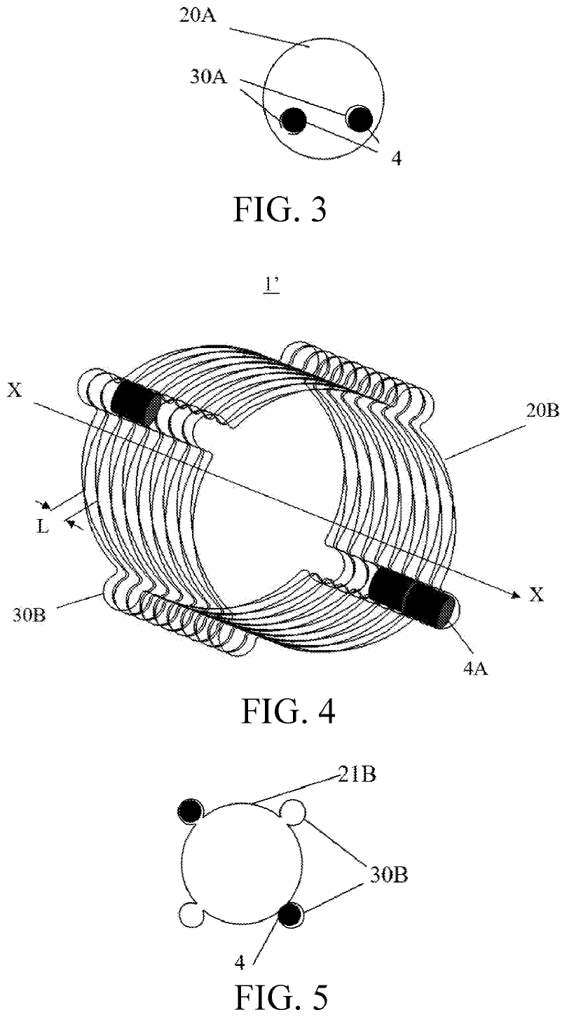

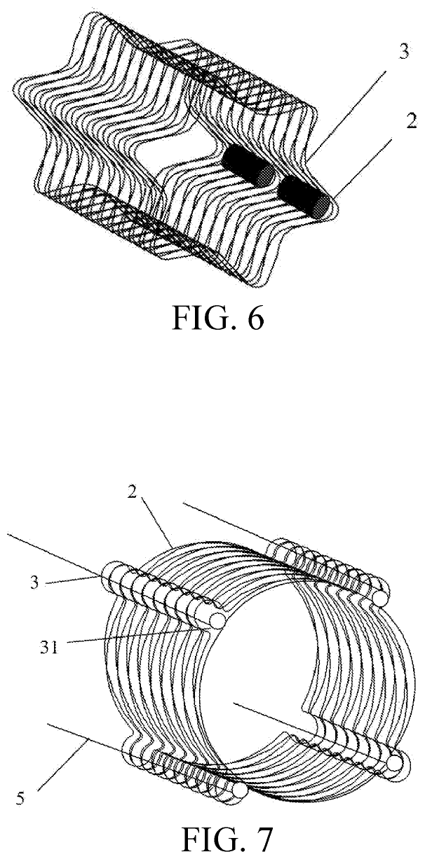

According to the intracavitary radiotherapy apparatus provided by this embodiment, only 2 groove bodies 30 are formed in the section located in front of the X axis (FIGS. 3), and 4 groove bodies 30 are formed in the section behind the X axis (FIG. 2). By means of this design, the size of the intracavitary radiotherapy apparatus in front of the X axis can be reduced, and the intracavitary radiotherapy apparatus is suitable for special focus distribution conditions.

### Embodiment III

As shown in FIG. 4, in the Embodiment III of the present invention, the radioactive particle groove 3 is formed in the surface of the body 2 in a protruding mode.

As shown in FIG. 4, an intracavitary radiotherapy apparatus 1 provided by the embodiment includes a body 2 and a radioactive particle groove 3, where radioactive particles 4 used for radiotherapy are arranged in the radioactive particle groove 3.

Specifically, the body 2 is formed by winding nickel-titanium alloy wires, is roughly a cylindrical wire mesh having a hollow cavity penetrating through same in the forward and backward directions, and has an axis X. Referring to FIG. 5, in the axial direction, the body 2 includes a plurality of body parts 20B which are formed by 4 arc-shaped nickel-titanium alloy wire body units 21B with the same radius in a roughly enclosing mode to form a circle, and the plurality of body parts 20B are arranged in parallel (roughly parallel). In other words, in the cross section shown in FIG. 5, the body units 21B are ¼ arc sections, and the four body units 21B are enclosed into an unclosed circular body part 20B. The plurality of body parts 20B are arranged in parallel along the axis X to form the body 2.

The radioactive particle groove 3 and the body 2 can be formed by weaving one or more nickel-titanium alloy wires by hot-pressing from the outer side of the body 2 to the X axis direction. The radioactive particle groove 3 is connected with the body 2 and is roughly a cylindrical wire mesh having a hollow cavity penetrating through same in the forward and backward directions along the Y direction. The Y axis of each radioactive particle groove 3 is parallel to the X axis of the body 2. Each radioactive particle groove 3 includes a plurality of groove bodies 30B. The plurality of groove bodies 30B are arranged in parallel along the Y axis. As shown in FIG. 5, each groove body 30B can be a semicircle or a ¾ circle. Each groove body 30B is connected with two adjacent body units 21B; and 4 groove bodies 30B are connected with 4 body units 21B at intervals to form a closed ring perpendicular to the X axis. The 4 radioactive particle grooves 3 can be uniformly distributed on the circumference formed by the body 2 (as shown in FIG. 4 and FIG. 5, the 4 groove bodies 30B are uniformly distributed on the circumference), and can also be non-uniformly distributed (for example, the 4 groove bodies 30B are non-uniformly distributed on the circumference).

In addition, the radioactive particle groove 3 is formed in the surface of the body 2 in a protruding mode. The schematic diagram of the section, perpendicular to the X axis, of the intracavitary radiotherapy apparatus 1 provided by this embodiment is shown in FIG. 5. In the same section, 4 body units 21B and 4 groove bodies 30B are provided. One or more radioactive particles 4 can be arranged in any one of radioactive particle grooves 3.

The diameter of the radioactive particle groove 3 is slightly greater than that of the radioactive particles 4, the diameter of the radioactive particle groove 3 can just allow the radioactive particles to penetrate through, but cannot be too large, and most preferably, the diameter of the radioactive particle groove 3 is 1.1-1.3 times that of the radioactive particles 4. Therefore, the radioactive particles can be prevented from getting loose and deviating from an expected position.

Similar to the Embodiment I, as shown in FIG. 1, the spacing L formed between the body units 21 which are adjacently arranged along the axis X is the width of the rhombus in the axial direction. The spacing L is less than the length of the radioactive particles 4, so that the radioactive particles 4 can be prevented from falling off from the gap of the rhombus. More preferably, the spacing L is less than half of the length of the radioactive particles 4. In other words, the length of the radioactive particles 4 is more than 2 times the spacing L, and the length of the radioactive particles 4 is greater than the width of two rhombuses in FIG. 1.

In addition, the radioactive particles 4 placed in the radioactive particle groove 3 are preferably placed at an intersection position (the intersection point in the width direction of the rhombus) of the adjacent body units 21B, so that the radioactive particles 4 can be fixed more firmly.

Similar to the Embodiment I, different numbers of radioactive particles 3 can be placed in 4 radioactive particle grooves 3 of the intracavitary radiotherapy apparatus 1 according to the design of the radiation dosimetry. Moreover, the positions of the radioactive particles 3 placed in the radioactive particle groove 3 can be changed according to the design of the radiation dosimetry. For example, a plurality of radioactive particles 3 in the same radioactive particle groove 3 are continuously placed (adjacent radioactive particles are placed end to end), or a plurality of radioactive particles 3 in the same radioactive particle groove 3 are placed at intervals (a large gap is formed between adjacent radioactive particles, and the radioactive particles are discontinuous). Through such position design, the radioactive particles are placed at intervals for treating diffuse tumors; and the radioactive particles are continuously placed for treating exogenous tumors.

According to the intracavitary radiotherapy apparatus provided by the present invention, due to the design of radioactive particle groove in channel mode, both the number and positions of the radioactive particles placed in the radioactive particle groove can be conveniently adjusted by a doctor before an interventional operation, so that compared with the design that the positions and number of the radioactive particles are fixed when a stent leaves a factory in the prior art, the intracavitary radiotherapy apparatus is more suitable for various focus distribution conditions.

### Embodiment IV

As shown in FIG. 5, the intracavitary radiotherapy apparatus provided by the present invention can be roughly pentagonal on the section perpendicular to the X axis, and includes 5 radioactive particle grooves 3C. Each radioactive particle groove 3C is roughly “V”-shaped (one corner of the pentagon). A body 2 formed by weaving fine metal wires made of titanium, nickel-titanium alloy or copper alloy has certain elasticity, so radioactive particles 4 can extend into a preset position along the X axis from the center of the pentagon during placement, then the radioactive particles are pushed from the center of the pentagon to the radial direction, and then the radioactive particles 4 are clamped into the radioactive particle grooves 3C.

### Embodiment V

As shown in FIG. 7, the outermost side of a radioactive particle groove of an intracavitary radiotherapy apparatus disclosed by this embodiment has a reduced radial size so as to prevent radioactive particles from falling off from an end port of the radioactive particle groove.

In FIG. 7, the end part of the radioactive particle groove 3 is provided with a shrinkage groove body 31, and the radial size of the shrinkage groove body is reduced to be half or less than that of a groove body 30 and is less than the width of the radioactive particles 4. The shrinkage groove body 31 and the groove body 30 can be formed by winding the same wire. Only one end of the end part of the radioactive particle groove 3 can be provided with the shrinkage groove body 31 (as shown in FIG. 7), or both ends are provided with the shrinkage groove bodies 31 (not shown in the figure).

The shrinkage groove body 31 can be provided with a marking ring for development (the marking ring can be seen by X rays and ultrasound) so as to guide a catheter to enter the radioactive particle groove 3 to accurately release the radioactive particles 4 into the radioactive particle groove 3. The catheter is a hollow catheter for feeding the radioactive particles or radioactive particle strips.

### Embodiment VI

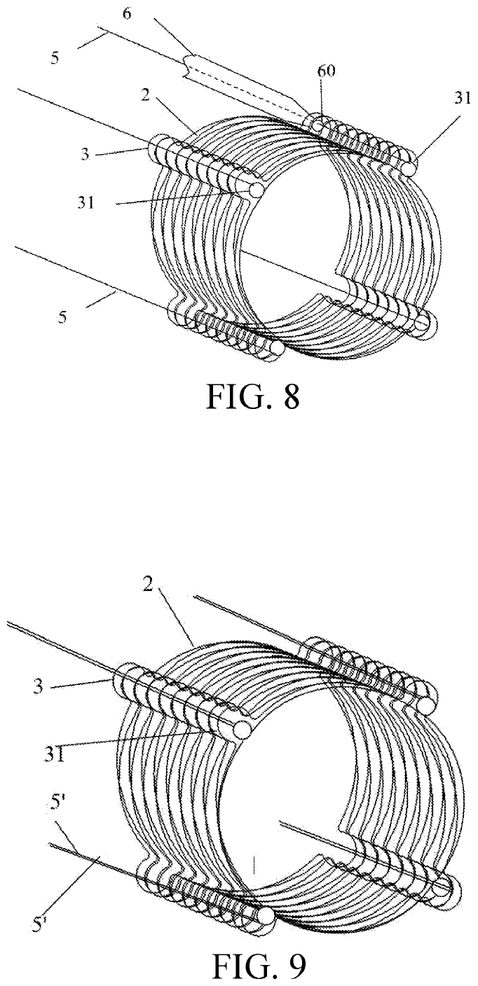

As shown in FIG. 7, an intracavitary radiotherapy apparatus disclosed by the embodiment further includes at least one guide wire 5. In the embodiment, as shown in FIG. 7 and FIG. 8, 4 guide wires 5 are provided, and a groove body 30 (or a shrinkage groove body 31) at the end part of each radioactive particle groove 3 is connected with one guide wire 5.

The guide wires 5 can be connected to a body 2 or the radioactive particle groove 3 for guiding a catheter to enter the intracavitary radiotherapy apparatus 1. The catheter sleeves the peripheries of the guide wires 5 and can move forward into the radioactive particle groove 3 along the guide wires 5. The connecting positions of the guide wires 5 and the body 2 or the radioactive particle groove 3 can be set arbitrarily according to actual requirements. The guide wires 5 can be made of the same material as the body 2, and can also be made of a softer material suitable for being placed in a human body.

As shown in FIG. 9, the guide wire 5 in this embodiment can also be of a structure penetrating through the shrinkage groove body 31. Specifically, one end of the guide wire 5 penetrates through the shrinkage groove body 31 along the interior of the groove body 3, then is wound back into the groove body 3, and finally is wound on the shrinkage groove body 31. Therefore, the guide wire in this embodiment can be of a single-wire structure (shown in FIG. 8) connected with the radioactive particle groove and can also be of a double-wire structure (shown in FIG. 9) penetrating through the shrinkage groove body.

In order to avoid vascular restenosis and stent thrombosis caused by implanting a metal stent into the human body, a drug capable of inhibiting cell proliferation is attached to the surface of the intracavitary radiotherapy apparatus 1 to accelerate endothelialization.

According to the intracavitary radiotherapy apparatus provided by the embodiment, the body and the radioactive particle groove are made of any one and/or more of nickel-titanium alloy, copper alloy and iron alloy.

The intracavitary radiotherapy apparatus provided by this embodiment is provided with the radioactive particle groove integrated with the body, has a more slender structure to cause a smaller wound, and can also enter a seriously blocked blood vessel; and since no radioactive particles are installed when the stent enters the human body, the stent without the particles is more flexible than a stent with the particles, so that the compliance is improved.

In addition, in the present invention, the stent is placed in the human body, and then the radioactive particles are accurately placed according to image display such as CT; and both the number and positions of the radioactive particles can be freely adjusted by a doctor according to an image, so that the number and the positions of the radioactive particles can be more accurate, and one solution corresponds one person (namely, different radioactive particle placement solutions are designed according to the focus condition of each patient).

Moreover, the manufacturing cost of the intracavitary radiotherapy apparatus can be reduced by the present invention. Due to the integrated design, sewing or welding or other process steps in the prior art are omitted, and therefore the manufacturing cost is reduced.

The present invention further provides a method for releasing radioactive particles to the intracavitary radiotherapy apparatus, including the following steps:

S1: the intracavitary radiotherapy apparatus is placed into a target position in a human body.

Similar to existing stent implantation, the intracavitary radiotherapy apparatus provided by the present invention is compressed into a catheter and then conveyed to the target position.

The intracavitary radiotherapy apparatus provided by the present invention is a stent woven by a single metal wire, and no radioactive particles are placed when the intracavitary radiotherapy apparatus is implanted, so that the intracavitary radiotherapy apparatus provided by the present invention has good expansibility and supporting force, and is not affected by the radioactive particles.

Compared with an intracavitary radiotherapy apparatus pre-loaded with radioactive particles, the intracavitary radiotherapy apparatus which is loaded with the radioactive particles later according to the present invention can be reduced to the minimum size, and therefore wounds and other side effects can be reduced in the implantation process. It is because that the intracavitary radiotherapy apparatus is pre-loaded with the radioactive particles, the size of the radioactive particles is increased on the periphery of the stent; and due to a fact that it is guaranteed that the position of the radioactive particles is fixed, the stent may be limited in shrinkage.

No radioactive particles are placed in the catheter, so the intracavitary radiotherapy apparatus using design that the radioactive particles are loaded later according to the present invention achieves better flexibility compared with the intracavitary radiotherapy apparatus pre-loaded with the radioactive particles. Because the stent pre-loaded with the radioactive particles is affected by the supporting force of the radioactive particles, the flexibility becomes poor, and the stent cannot be implanted into a bent blood vessel easily.

S2: A catheter loaded with radioactive particles or particle strips is pushed into a radioactive particle groove.

The catheter loaded with the radioactive particles inside is pushed into the radioactive particle groove 3 by using a marking ring on the shrinkage groove body 31 visible under X rays. If the intracavitary radiotherapy apparatus is provided with a guide wire 5 (shown in FIG. 7) connected to a far-end shrinkage groove body 31, the catheter can enter the radioactive particle groove 3 along the guide wire 5 by using the guide wire 5.

The catheter 6 is a hollow pipe, and the radioactive particles 4 (shown in FIG. 1) or radioactive particle strips 4A (shown in FIG. 4) formed by connecting a plurality of radioactive particles in series can be loaded in the catheter. The outer diameter of the far end 60 of the catheter 6 is less than the diameter of the radioactive particles, so that the radioactive particles can be prevented from slipping from the catheter, and the far end of the catheter can enter the radioactive particle groove 3. Moreover, the diameter of the catheter 6 is gradually increased to be greater than or equal to the inner diameter of the groove body 30 from a far end 60 to the near end (not shown in the figure).

Firstly, the far end 60 of the catheter enters the interior of the radioactive particle groove 3 (as shown in FIG. 8). Secondly, the far end 60 of the catheter 6 gradually advances along the Y axis of the central axis of the groove body 30 (the dotted line in FIG. 8) and goes deep into the interior of the radioactive particle groove 3; the diameter of the part, entering the interior of the radioactive particle groove 3, of the catheter 6 is gradually increased; and the hardness of the catheter 6 is enough to expand the groove body 30 and the shrinkage groove body 31 from inside to outside.

If the intracavitary radiotherapy apparatus is provided with the guide wire as shown in FIG. 8 and FIG. 9, the guide wire arranged in the radioactive particle groove can be used for guiding the catheter to enter the radioactive particle groove. If the guide wire (shown in FIG. 1) is not available, the catheter enters the radioactive particle groove of the intracavitary radiotherapy apparatus by means of developing equipment (such as X rays) and a marking ring (visible under the X rays) on the radioactive particle groove. The guide wire is of a single-wire structure connected with the radioactive particle groove or a double-wire structure penetrating through the shrinkage groove body. In the double-wire structure, the guide wire is wound in the groove body 30, so that after the catheter 6 enters the groove body 30 along the guide wire 5, the guide wire 5 can be easily drawn out of the human body from the groove body 30 and the catheter 6. How to guide the catheter by using the guide wire has been known in the prior art and is not repeated herein.

S3: Radioactive particles or particle strips are pushed into a first radioactive particle groove from a catheter.

When the far end 60 of the catheter 6 reaches a preset position, one radioactive particle 4 in the catheter 6 is pushed out by a push rod. After one radioactive particle is pushed out, the far end 60 of the catheter 6 will reversely move by a preset distance along the Y axis until the far end reaches a position where the next radioactive particle is to be placed. Then, another radioactive particle is pushed, and the far end moves backwards. The operation is repeated until all the radioactive particles in the radioactive particle groove are placed.

The far end 60 (namely the front end extending into the groove body 30) of the catheter has certain elasticity and enables the radioactive particles 4 to be extruded out from an opening of the far end 60 so as to fall into the groove body 30.

When the far end 60 moves backwards, the groove body 30 pushed out by the catheter 6 and the shrinkage groove body 31 will shrink. Because radial size of the groove body 30 and the radial size of the shrinkage groove body 31 are less than or equal to the diameter of the radioactive particles 4, the radioactive particles placed inside can be clamped, and therefore the effect of fixing the radioactive particles 4 is achieved.

S4: The catheter is withdrawn from a first radioactive particle groove.

S5: The catheter is pushed into a second radioactive particle groove.

The steps S3-S5 are repeated until all radioactive particles or particle strips are placed into corresponding radioactive particle grooves, and then the catheter is withdrawn.

By using a puncture needle, the radioactive particles are implanted into the human body during an operation, so that both the positions and number of the radioactive particles can be adjusted freely according to the state of an illness. Particularly, by using the apparatus and the method provided by the present invention, the doctor can finely adjust the positions of the radioactive particles according to a real focus situation during the operation by referring to a preoperative treatment plan.

Those skilled in the art may understand that the single particle can be placed in the radioactive particle groove of the intracavitary radiotherapy apparatus, and particle strips containing multiple particles can also be placed in the radioactive particle groove. Moreover, the particle strips or the particles can be completely accommodated in the radioactive particle groove (that is, the particle strips or the particles do not exceed the radioactive particle groove in the axial direction in the radioactive particle groove) or a small part of the particle strips or the particles can be located outside the radioactive particle groove (that is, a part of the particle strips or the particles are located outside the radioactive particle groove in the axial direction in the radioactive particle groove), and it is only to guaranteed that the particles or the particle strips cannot disconnect. Under the condition that a small part of the radioactive particles or the particle strips are located outside the radioactive particle groove, the radioactive particles or the particle strips can play a role in radiotherapy on lesions outside the radioactive particle groove.

The intracavitary radiotherapy apparatus provided by the present invention adopts the integrated design, so that the size thereof can be reduced to a smaller size than that of a split design, thereby facilitating implant into the human body. Moreover, the radioactive particles can be conveniently replaced or increased (for example, when both the positions or number of the radioactive particles cannot meet the requirement, new radioactive particles can be placed in the apparatus again except the placed radioactive particles; and if particle strips are placed in the apparatus, the particle strips can be sucked out and replaced with new particle strips).

In conclusion, the intracavitary radiotherapy apparatus provided by the present invention is provided with the radioactive particle groove integrated with the body, has a more slender structure to cause a smaller wound, and can also enter a seriously blocked blood vessel; and compared with a stent pre-loaded with particles in the prior art, the apparatus is flexible, the situation that the stent becomes “hard” due to the fact that the particles are loaded in advance, and the apparatus difficultly enters the bent blood vessel is avoided, so that the compliance is improved. In addition, because the stent is placed in the human body firstly, then the radioactive particles can be accurately placed according to CT and other image display and the focus condition around the stent, both the number and the positions of the radioactive particles can be freely adjusted by the doctor according to the image, so that the number and the positions of the radioactive particles can be more accurate. Moreover, the manufacturing cost of the intracavitary radiotherapy apparatus can be reduced by the present invention. Due to the integrated design, sewing or welding or other process steps in the prior art are omitted, and therefore the manufacturing cost is reduced.

The intracavitary radiotherapy apparatus provided by the present invention and the use method therefor are illustrated in detail above. For those skilled in the art, without departing from the essence of the present invention, any obvious changes of the present invention will constitute an infringement of the patent right of the present invention and will bear corresponding legal liabilities.

1. An intracavitary radiotherapy apparatus, which is used for carrying radioactive particles or particle strips, comprising:
a body that is formed by winding metal wires and is a wire mesh having a hollow cavity penetrating through same in the forward and backward directions; and
a radioactive particle groove that is formed in the outer surface of the body, is a cylindrical wire mesh having a hollow cavity penetrating through same in the forward and backward directions, and is used for accommodating the radioactive particles or particle strips, wherein
the radioactive particle groove comprises a plurality of groove bodies arranged in parallel, and spacing between every two adjacent groove bodies is less than the length of the radioactive particles or the particle strips; and
the radioactive particle groove and the body are made of the same material by winding metal wires, or are made of biodegradable material by means of one-step injection molding.

2. The intracavitary radiotherapy apparatus according to claim 1, wherein
the radioactive particle groove is formed by inwards or outwards hot-pressing from the outer surface of the body after winding.

3. The intracavitary radiotherapy apparatus according to claim 1, wherein
the spacing between every two adjacent groove bodies is the same as that between body units.

4. The intracavitary radiotherapy apparatus according to claim 2, wherein
the diameter of the radioactive particle grooves is 0.8-1.2 times that of the radioactive particles.

5. The intracavitary radiotherapy apparatus according to claim 1, wherein
the radioactive particle groove is formed in the surface of the body in a protruding mode, and the inner diameter of the radioactive particle grooves is less than or equal to the diameter of the radioactive particles or particle strips.

6. The intracavitary radiotherapy apparatus according to claim 1, wherein or 12the radioactive particle groove is formed in the surface of the body in a concave mode, and the inner diameter of the radioactive particle grooves is less than or equal to the diameter of the radioactive particles or particle strips.

7. The intracavitary radiotherapy apparatus according to claim 1, wherein
the number of the radioactive particle grooves is different in the axial direction of the intracavitary radiotherapy apparatus.

8. The intracavitary radiotherapy apparatus according to claim 1, wherein the intracavitary radiotherapy apparatus further comprises guide wires, and the guide wires are fixed to
the body or the radioactive particle grooves and are of a single-wire or double-wire structure.

9. A method for using the intracavitary radiotherapy apparatus according to claim 1, comprising the following steps:
S1: placing the intracavitary radiotherapy apparatus at a target position in a human body;
S2: pushing a release catheter carrying radioactive particles or particle strips into the radioactive particle groove;
S3: pushing the radioactive particles or particle strips into the radioactive particle groove from the release catheter; and
S4: withdrawing the release catheter from the radioactive particle groove.

10. The method for using the intracavitary radiotherapy apparatus according to claim 9, wherein
the radioactive particle groove at least comprises a first radioactive particle groove and a second radioactive particle groove,
in the steps S1-S4, the radioactive particles are placed in the first radioactive particle groove;
then, the release catheter is pushed into the second radioactive particle groove; and
the steps S3-S5 are repeated until all the radioactive particles or particle strips are placed in the corresponding radioactive particle grooves and the release catheter is withdrawn.

11. The method for using the intracavitary radiotherapy apparatus according to claim 10, further comprising the following step:
guiding the release catheter to enter the radioactive particle groove by the guide wire arranged in the radioactive particle groove.

12. The method for using the intracavitary radiotherapy apparatus according to claim 11, wherein the guide wire is of a single-wire structure connected with the radioactive particle groove or a double-wire structure penetrating through a shrinkage groove body.
